(19) United States
(12) Patent Application Publication
(10) Pub. No.: US 2009/0111207 A1
(43) Pub. Date: Apr. 30, 2009

(54) METHOD OF FABRICATING AN INTEGRATED DETECTION BIOSENSOR

(76) Inventors: Houtai Choumane, Fresnes (FR); Claude Weisbuch, Paris (FR); Henri Benisty, Palaiseau (FR); Khoi-Nguyen Ha, Bagneux (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/090,559

(22) PCT Filed: Oct. 17, 2006
(86) PCT No.: PCT/FR2006/002327

§ 371 Date: Dec. 9, 2008

(30) Foreign Application Priority Data
Oct. 18, 2005 (FR) 0510614

(51) Int. Cl. H01L 21/02; A61B 5/1455

(52) U.S. Cl. 438/70; 600/310; 438/72; 257/E21.002

(57) ABSTRACT

A method of fabricating an integrated detection biosensor, the biosensor comprising an assembly (10) of photodetectors (12) of CCD or CMOS type on which there is deposited or formed a filter for rejecting excitation light λe, the filter comprising at least one absorbent layer (14) together with a Bragg mirror or an interference filter, forming a support for chromophore elements that are to be illuminated by the excitation light λe.

The invention relates to a method of fabricating an integrated detection biosensor, and to the biosensor obtained by performing the method.

An integrated detection biosensor comprises a substrate supporting chromophore elements and an assembly of photodetectors for picking up the light emitted by the chromophore elements in response to light excitation, the assembly of photodetectors being associated with the substrate and forming a unitary assembly therewith.

Document WO 02/16912 discloses a biosensor of that type in which an interference mirror and an absorbent layer are arranged in the substrate to reject the chromophore-excitation light and to prevent delivering noise to the photodetectors provided on the rear face of the substrate. Document WO 2004/042376 also discloses an integrated luminescence biosensor with evanescent excitation, in which the substrate can be associated with an assembly of photodetectors and includes on its surface a planar waveguide containing photoluminescent ingredients that are illuminated by primary excitation light and that themselves emit light for exciting chromophores deposited on the waveguide.

Those structures have the advantage of improving the sensitivity of detection by very significantly increasing the effectiveness with which the light emitted by the chromophores is collected, and by reducing the extent to which excitation light is captured, and also reducing interfering fluorescence coming from the surrounding medium: it is known that about 80% of the light emitted by the chromophores is transmitted into the substrate, and that a lens associated with a matrix of charge-coupled device (CCD) photodetectors placed over the chromophores in air can pick up only a small fraction of the 20% of the light flux that is emitted into the air. As a result, the maximum detection sensitivity is typically of the order of 10 chromophores per square micrometer (μm²). Placing a set of photodetectors on the rear face of the substrate or on the face opposite from that carrying the chromophores enables the light flux emitted by the chromophores to be collected with effectiveness that is several tens of times greater than that of a standard imager placed above the chromophores.

Document US 2002/081716 and WO 2004/059006 disclose integrated detection biosensors having optical filters that stop the wavelength of the light used for exciting the chromophores while passing the fluorescence emitted by the chromophores, however those filters are made out of materials that are autofluorescent and the light they emit is sufficient to mask the fluorescence emitted by the chromophores. That drawback is made worse when the excitation light has a wavelength in the ultraviolet, as described in those two prior art documents.

An object of the present invention is to avoid those drawbacks and to further improve the integrated detection biosensor described in Document WO 02/16912.

To this end, the invention provides a method of fabricating an integrated detection biosensor, the biosensor comprising a substrate for carrying chromophore elements that emit light in response to light excitation at a given wavelength, and an assembly of photodetectors associated with the substrate to pick up the light the chromophore elements emit towards the inside of the substrate, the method being characterized in that it consists in depositing thin layers on the assembly of photodetectors, the thin layers constituting the above-mentioned substrate together with a filter both for omnidirectional rejection of the chromophore element excitation light and for transmission of the light emitted by said elements, the filter providing transmission for the excitation light of about 10⁻⁶ or less, preferably about 10⁻⁸, and presenting an autofluorescence level of 10⁻⁶ or less.

To further limit the autofluorescence of the filter, it is advantageous to use excitation light having a wavelength in the visible spectrum or in the near infrared.

The method of the invention makes it possible to make an ultrasensitive detection biosensor that is integrated and that does not include a lens or optical component, and in which biological probes can be deposited directly on a thin layer rejection filter covering an assembly of photodetectors. It is thus possible to make miniature biosensors at low cost by using known techniques for mass producing microelectronic components, such biosensors also presenting sensitivity of the order of one chromophore/μm².

In a first embodiment of the invention, the rejection filter comprises at least one thin layer that is absorbent at the excitation wavelength of the chromophore elements.

The absorbent layer is provided to absorb the excitation light omnidirectionally independently of the angle at which the biosensor is illuminated or the angle at which the excitation light is diffused.

This absorbent layer may be made by any known means, e.g. by the sol-gel method or by depositing and spreading a layer of dye possibly dispersed in an inorganic or polymer matrix, using a method of the spin coating type or a method of the dip coating type.

In another embodiment of the invention, the rejection filter comprises, in combination with the absorbent filter, a Bragg mirror made up of thin layers of materials that are transparent at the chromophore emission wavelength, or an interference filter made for example of superposed thin polymer layers.

The Bragg mirror or the interference filter covers at least one absorbent layer that is deposited on the assembly of photodetectors.

It is the combination of a Bragg mirror or an interference filter together with an absorbent layer that is capable of giving best results in terms of rejecting the light used for exciting chromophore elements. The Bragg mirror produces an effect of amplifying the excitation by constructive interference and an effect of pure rejection of the excitation (directional effect), with the rejection nevertheless being provided mainly by the absorbent layer. The rejection by the Bragg mirror provides additional reduction in the level of fluorescence in the absorbent layer.

In a variant, the biosensor includes an opaque surface layer, e.g. made of metal, having holes formed therein, this layer serving to limit the overall light flux on the biosensor.

In order to reduce the autofluorescence of the molecules that absorb the excitation light in the absorbent layer, the invention makes provision in one embodiment to form, on the assembly of photodetectors, a plurality of superposed absorbent thin layers of different kinds, in which a lower layer (closer to the photodetectors) serves to absorb autofluorescence from a higher layer.

This disposition in cascade of absorbent thin layers is particularly advantageous when the spectrum difference between the excitation wavelength for the chromophore elements and the center wavelength of the light emitted by the chromophore elements is large.

In any event, the materials selected for the absorbent layer(s) are fundamental for good operation of the biosensor.

In an embodiment of the invention, the rejection filter comprises a Bragg mirror made up of a series of superposed thin layers presenting optical thickness equal to one-fourth of the excitation wavelength, the Bragg mirror providing rejection of 0.025 of the excitation (i.e. 0.1 pure rejection). In this structure, interference effects at the surface of the substrate enable the energy of the excitation electromagnetic field to be increased by a factor of about 4, thereby leading to an amplification in the photo-excitation rate by a factor of 4. Concerning the transmission of excitation energy through these layers, that corresponds to an equivalent optical density of 1.6. The Bragg mirror is associated with an absorbent layer having optical density of 6.4, and an autofluorescence level of less than 10^{-6.4} times the intensity of the exciting light, the rejection filter presenting total equivalent optical density equal to 8, giving rise to a rejection rate of 10⁻⁸. Detection sensitivity is then one chromophore element/μm² for the usual chromophores.

The biosensor of the invention can be made by depositing one or more absorbent thin layers on a matrix of photodetectors, and then (optionally) depositing thin layers for forming a Bragg mirror or an interference filter, the various layers being deposited or formed in succession one on another.

In a variant embodiment, the method of the invention consists in making the rejection filter on an initial substrate, then in depositing the assembly formed by the filter and the initial substrate on an assembly of photodetectors, the filter lying between said assembly of photodetectors and the initial substrate, and finally in removing the initial substrate.

Under such circumstances, the rejection filter is fastened to the assembly of photodetectors by adhesion, either because of its own adhesion, or by means of a layer of an appropriate adhesive material.

The rejection filter, which is initially formed on the initial substrate, comprises an absorbent film, or a reflective film, or the association of an absorbent film with a reflective film. Compared with a method of fabricating the various layers directly on the sensor, this overcomes constraints associated with various treatment or annealing operations, thereby making a broader range of design and integration possibilities available.

This makes it possible in particular to begin by forming a reflective film such as a Bragg mirror on the initial substrate, involving annealing operations that would not be tolerated well by the photodetectors and by the absorbent film, and in subsequently depositing the thin layer or the assembly of thin layers forming the absorbent film on the Bragg mirror.

Advantageously, the rejection filter and the initial substrate can form a flexible film that is easy to store and to use, e.g. in the form of a roll.

In a variant, the Bragg mirror or the interference filter may be formed on an initial substrate, and the absorbent film may be formed on another initial substrate, thus making it possible subsequently to make the biosensor of the invention by transferring the absorbent film onto an assembly of photodetectors, and then by transferring the Bragg mirror or the interference filter onto the absorbent film. Once the biosensor is made, probes optionally including fluorescent markers are deposited in the liquid phase on determined zones, e.g. in an array, on the rejection filter of the biosensor (a technique known as “spotting”). After drying, the biosensor is stored, and its storage duration can be long. The probes may comprise fluorescent markers.

Optionally, an aqueous buffer liquid containing a wetting agent is used for depositing the probes on the rejection filter, the surface of which can be highly hydrophobic.

In an embodiment of the invention, the biosensor carrying the probes is finally encapsulated in a package that is subsequently usable for hybridizing probes, the package having at least one liquid inlet and one liquid outlet that are interconnected inside the package by a channel extending over the surface of the filter carrying the probes, at least one window for observing and/or illuminating the probes by excitation light being formed in the face of the package that covers the probes.

The opposite face of the package, situated beside the photodetectors, gives access to an electronic interface for connecting the photodetectors with data processing means.

Typically, the assembly of photodetectors used is a matrix of photodetectors of the charge-coupled device (CCD) or complementary metal oxide on silicon (CMOS) type, having its front face covered by the rejection filter.

In a variant, it can be advantageous to use a matrix of CCD or CMOS photodetectors that is illuminated by its rear face, in order to improve sensitivity by a factor of 2.

In the visible spectrum, matrices of CCD photodetectors illuminated via the front face (beside the photodetectors) present sensitivity that is reduced by about half compared with the sensitivity of matrices of photodetectors illuminated via the rear face, because of photons being absorbed by their polysilicon transfer grids. Conversely, using illumination via the rear face requires the silicon substrate to be thinned, an operation that is difficult.

According to another characteristic of the invention, openings are formed in one or more of the layers of the rejection filter in register with some of the photodetectors in order to calibrate the extent to which the excitation light is rejected by said layers.

The invention can be better understood and other characteristics, details, and advantages thereof appear more clearly on reading the following description made by way of example and with reference to the accompanying drawings, in which:

FIG. 1 is a fragmentary diagrammatic section view of a biosensor of the invention;

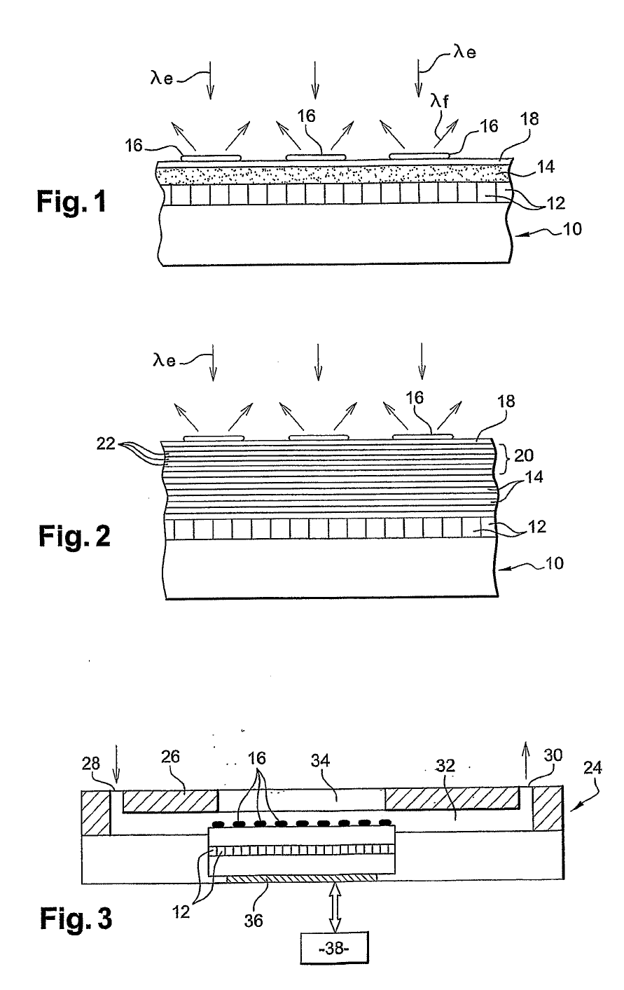

FIG. 2 is a fragmentary diagrammatic section view of a variant embodiment of the biosensor;

FIG. 3 is a diagrammatic section view showing a biosensor of the invention mounted in a hybridization box;

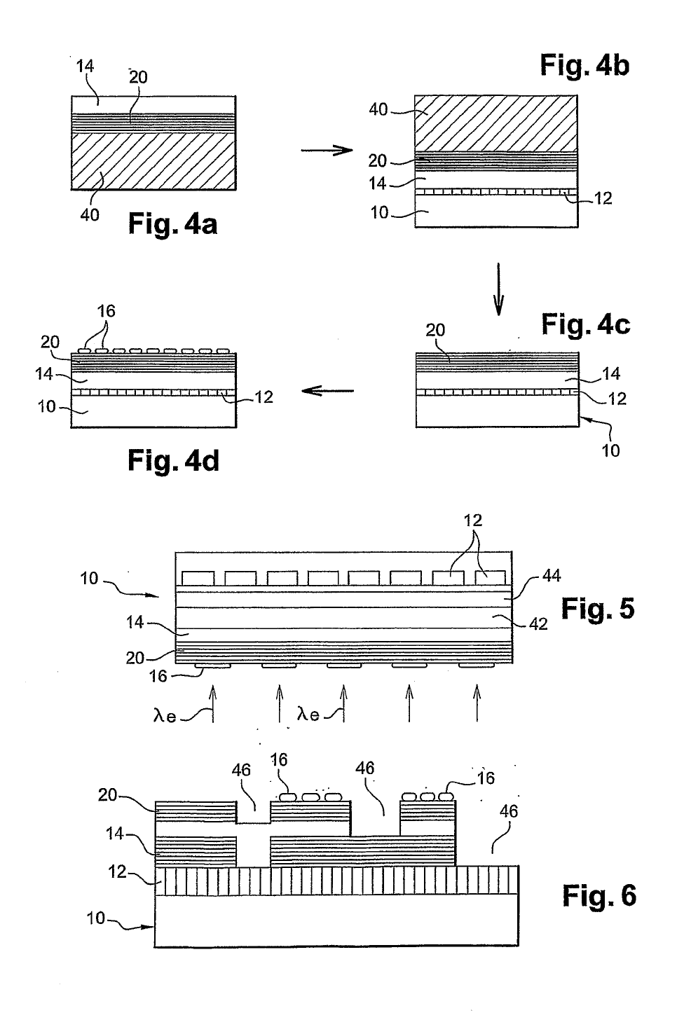

FIG. 4 is a diagram showing four steps in making a biosensor of the invention;

FIG. 5 is a diagrammatic fragmentary view in section of a biosensor of the invention having a CCD photodetector matrix illuminated through the rear face;

FIG. 6 is a fragmentary diagrammatic section view of a biosensor constituting another variant of the invention;

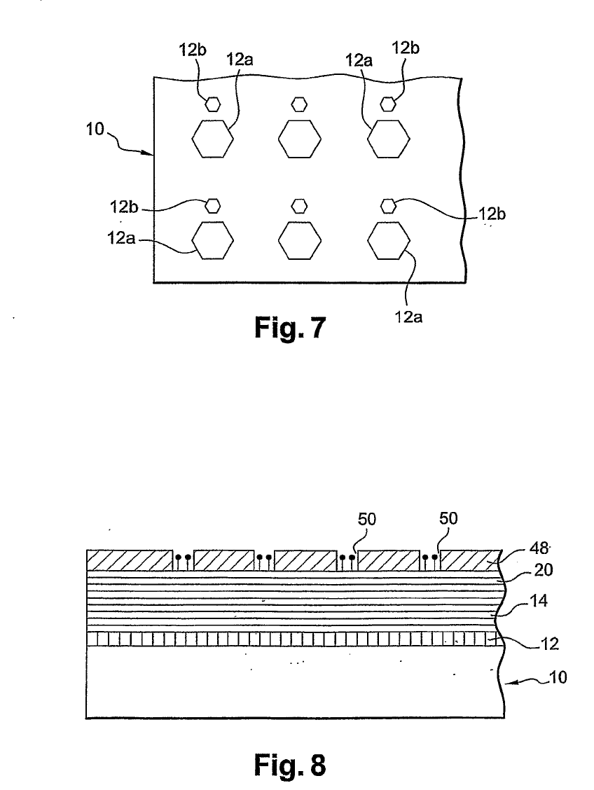

FIG. 7 is a fragmentary diagrammatic view of a matrix of photodetectors having pixels of different sizes; and

FIG. 8 is a diagrammatic view in section of a variant embodiment of the invention.

The biosensor of FIG. 1 comprises a matrix assembly 10 of photodetectors 12 of the CCD or CMOS type, having deposited thereon a layer 14 of a material for absorbing light radiation for exciting chromophore elements, located in spots 16 on the surface of the biosensor, where the chromophore elements emit light centered on a wavelength λf when excited by light radiation having a wavelength λe (for example λf may be equal to 570 nanometers (nm) and λe may be equal to 532 nm, when the chromophore elements are Cy3 markers), the excitation wavelength being selected to be in the visible spectrum (about 400 nm to 750 nm) or in the near infrared (about 750 nm to 2500 nm).

About 80% of the light flux emitted by the chromophore elements passes into the absorbent layer 14 and is captured by the photodetectors 12, the excitation light flux at the wavelength λe being absorbed by the layer 14. This layer preferably presents optical density of not less than 6 at the wavelength under consideration, so as to ensure a detection sensitivity level of 1 chromophore element per μm². The absorbent layer 14 may be formed by a single layer of absorbent material, or by a plurality of superposed absorbent layers of different kinds for reducing the autofluoresence of said layer as caused by the excitation light. Under such circumstances, an absorbent layer n situated under an absorbent layer n+1 presents a nature that is determined for absorbing the autofluorescence of the absorbent layer n+1 while passing the light flux at the wavelength λf to the photodetectors 12.

Because of the absence of an imager element between the chromophores and the photodetectors, a single photodetector can receive light signals coming from different points or zones, thereby generating an interfering signal or crosstalk, which becomes greater with greater spacing or vertical distance between these points or zones and the photodetector. In a preferred embodiment of the invention, this spacing is small, so the interference signal is reduced to a minimum. For example, the diameter of these points or zones is 400 micrometers (μm) and their spacing relative to the photodetectors is 10 μm, such that the interfering signal is minimized. When the spacing relative to the photodetectors is greater, and reaches 100 μm, then the crosstalk signal can be large and of a kind that will reduce detection sensitivity. Under such circumstances, computer deconvolution processing can be performed on the image with interference, as is well known to the person skilled in the art, in order to recover the useful signal by eliminating the interfering signal.

The absorbent layer 14 can be prepared and deposited on the photodetectors 12 as follows:
A solution is prepared of a dye having rejection compatible with the light emission of the fluorescence markers used, i.e. a dye that stops the excitation light but passes a portion of the emission spectrum of the markers. Dyes satisfying these criteria comprise metallic complexes based on chromium or on cobalt, with binders formed by organic molecules based on azo derivatives.

In a variant, it is possible to use a mixture of a dye (having a function of absorbing the excitation light) and some other component that eliminates or stops fluorescence of the absorbent molecule.

The dye solution is prepared by dissolving one gram (g) of dye in one milliliter (mL) of dimethyl formamide (DMF). After stirring, the resulting solution is filtered and mixed with 1.5 mL of a polyimide solution (as sold by HD Microsystems under the reference PI 2555) or with butylcyclobenzene. The final solution has a concentration by weight of dye of about 400 milligrams per milliliter (mg/mL) and a molar extinction coefficient equal to about 9×10³ per centimeter (cm⁻¹).
The dye solution is deposited on the photodetectors of a CCD matrix sensor, from which the protective window has been removed and in which the metallic contacts have been protected, e.g. by localized deposition of a coating resin capable of providing good sealing, good mechanical strength, and good chemical resistance during the thermal annealing or polypolymerization steps that are needed for making the biosensor. For example, it is possible to use the EPOTEK T7139 resin from the supplier Polytec PI SA. The dye solution is spread by a spin coating technique at a speed of rotation of 3000 revolutions per minute (rpm), with spreading being followed by pre-annealing at 100° C. for 30 minutes (min) in a stove, followed by annealing at 210° C. in a stove for 1 hour (h) 30 min, these temperatures being acceptable for the matrix of photodetectors.

The resulting dye film has a thickness of about 10 μm, and an optical density equal to 9 at the wavelength of 532 nm, which corresponds to transmission of 10⁻⁹.

Biological probes are subsequently deposited on the surface of the absorbent layer 14 by a so-called “spotting” technique so as to form the above-mentioned spots 16. Since the absorbent layer 14 is naturally very hydrophobic, it is necessary, in order to be able to deposit the biological probes, to make use of a buffer liquid that contains a relatively high quantity of a wetting agent of the sodium dodecyl sulfate (SDS) type in order to form spots 16 having a dimension of about 400 μm (or less as a function of the application). In this context, it should be observed that the relative dimensions of the various elements shown in FIG. 1 are not complied with in the drawing, for reasons of clarity. In reality, the spots 16 have a dimension of 100 μm to 400 μm for example, the absorbent layer 14 has a thickness of about ten μm, the photodetectors 12 has unit dimensions of the order of ten μm, so the spots 16 cover one or more tens of photodetectors.

In general, a functionalization layer 18 is formed on the top surface of the layer 14 on which the biological probes are deposited, this functionalization layer serving to fix the probes.

In the variant embodiment of FIG. 2, the photodetectors 12 of the sensor 10 are covered both by a plurality of absorbent thin layers 14 of different kinds, enabling the influence of the autofluoresence of the dyes used in the absorbent layers to be reduced, and by a Bragg mirror 20 formed by a plurality of superposed thin layers 22 of dielectric material, the layers having refractive indices that are respectively high and low and that are placed in alternation, in a manner that is well known to the person skilled in the art.

By way of example, use can be made of alternating layers 22 of material having refractive indices equal to 1.45 (low index) and 1.95 (high index), with the number of layers depending on the ratio of these two indices, and being equal to 20, for example, when the desired optical density for the Bragg mirror is equal to 1.

These alternating thin layers 22 have optical thickness equal to one-fourth of the excitation wavelength λe, thereby increasing the excitation light flux intensity at the chromophore elements by a factor of 4 by means of constructive interference, and thus increasing the intensity of the light emitted by the chromophore elements in response to this excitation.

In addition, these layers reduce the intensity of the light excitation on the absorbent filter, thus reducing the autofluoresence of the filter.

The Bragg mirror 20 need not be centered exactly on the excitation wavelength λe, so as to increase the total rejection slope of the filter constituted by the Bragg mirror 20 and by the multilayer absorbent 14. Nevertheless, it is necessary for the Bragg filter to be centered in relatively accurate manner on the excitation wavelength λe in order to achieve significant amplification of the light excitation (amplification greater than 3).

The alternating thin layers 22 of the Bragg mirror 20 can be deposited using any known method, e.g. by physical deposition techniques, by a sol-gel method, or indeed by extrusion. Thereafter, a functionalization layer 18 is formed on the top surface of the Bragg mirror 20, and then spots 16 containing the biological probes can be deposited and fixed on said layer 18 as described above for the biosensor of FIG. 1. In a variant embodiment, a semitransparent metal mirror can act as a first filter.

In a variant embodiment, the Bragg mirror 20 deposited on the absorbent layers 4 may be replaced by an interference filter made up of superposed thin layers of polymers having alternating high and low refractive indices, the techniques of fabricating such interference filters being known to the person skilled in the art and described in particular in U.S. Pat. No. 6,737,154.

The biosensor of the invention, on which the spots 16 have been formed containing the biological probes, can finally be encapsulated in a package or a hybridization cartridge 24 (FIG. 3) having a front face 26 including at least one liquid inlet opening 28 and at least one liquid outlet opening 30 interconnected by a channel 32 enabling liquid entering via the opening 28 to flow over the face of the biosensor carrying the spots 16 in which biological probes have been deposited.

The front face 26 of the cartridge 24 includes at least one other opening 34 formed facing the biological probe deposition spots 16 and enabling these spots to be illuminated by the light for exciting the chromophore elements.

An electronic interface 36 associated with the rear face of the assembly 10 of photodetectors is accessible via the rear face of the hybridization cartridge 24 and enables the data picked up by the photodetectors to be transferred to data processor means 38.

When the rejection filter that covers the assembly 10 of photodetectors includes a Bragg mirror 20 and absorbent layers 14, difficulties can be encountered during fabrication of the Bragg mirror insofar as that requires annealing at a relatively high temperature in order to densify the layers 22 and prevent subsequent deformation thereof, whereas the assembly 10 of photodetectors and the dyes used in the absorbent layers 14 generally need to be protected from high temperatures.

To avoid those drawbacks, the invention provides a method of fabricating the biosensor that comprises essential steps a, b, c, and d shown in FIG. 4, the method consisting, in step a, in forming initially the Bragg mirror 20 (or an interference filter) on an initial substrate 40 of conventional type, then in depositing or forming the absorbent layer(s) 14 on the Bragg mirror. This enables the layers 22 of the Bragg mirror (or the interference filter) to be subjected to the necessary annealing without worrying about the influence of this annealing on the other components of the biosensor.

Thereafter, in step b, the assembly formed by the substrate 40, the Bragg mirror 20, and the absorbent layer 14 is transferred onto the assembly 10 of photodetectors by being turned over so that the absorbent layer 14 is pressed against the photodetectors 12 of the assembly 10.

In the following step c, the initial substrate 40 is removed so as to obtain a biosensor of the type shown in FIG. 2.

Following step d consists in depositing on the Bragg mirror 20 the spots 16 that contain the biological probes.

In a variant, it is naturally possible to form the Bragg mirror 20 (or the interference filter) on an initial substrate 40, and the absorbent layer(s) 14 on another initial substrate, and then to deposit them in turn on the assembly 10 of photodetectors, putting the absorbent layer(s) 14 into place initially on the photodetectors 12 and removing the initial substrate carrying the absorbent layers, and then depositing the Bragg mirror 20 on the absorbent layer(s) 14, and finally removing the initial substrate 40 carrying the Bragg mirror.

It is also possible to form or deposit the absorbent layer(s) 14 directly on the photodetectors 12 of the assembly 10, while in parallel forming a Bragg mirror 20 on an initial substrate 40, and then to turn over the resulting assembly in order to deposit the Bragg mirror 20 on the absorbent layer(s) 14 carried by the assembly 20 of photodetectors, and then remove the initial substrate 40.

The technology for fabricating interference filters by stacking layers of polymers, as described in U.S. Pat. No. 6,737,154, is well adapted to this method of fabrication by transfer, with the absorbent layer(s) and the interference filter being secured by adhesive.

In yet another variant of this method, it is the absorbent layers 14 that are formed initially on an initial substrate 40, followed by an interference filter or a Bragg mirror that is formed on the absorbent layers, after which the assembly comprising the absorbent layers 14 and the Bragg mirror or interference filter is removed from the initial substrate 40 and deposited on and bonded to the assembly 10 of photodetectors. Under such circumstances, after the surface has been functionalized, it is possible to form the spots 16 containing the biological probes on the Bragg mirror 20 or on the interference filter, prior to transferring said assembly onto the assembly 10 of photodetectors.

This technology makes it possible to make the absorbent layer(s) 14 and the Bragg mirror 20 or the interference filter in the form of films that are can be flexible films deposited on an initial substrate 40 that is likewise constituted by a flexible film. The assembly comprising the initial substrate 40, the interference filter or the Bragg mirror 20, and the absorbent filter 14 then constitutes a flexible film that can be stored in the form of a roll. Where necessary, particles or nanofibers such as, for example: fullerenes, carbon nanotubes, glass fibers, . . . , can be incorporated in the film to reinforce its mechanical properties. Conversely, it is possible to incorporate polymer inclusions of micrometer size in the film, the inclusions having a glass transition temperature that is higher than ambient temperature such that the film can be made flexible by being heated at the time the initial substrate 40 is removed and then return to being rigid when deposited on the assembly 10 of photodetectors.

In all embodiments of the biosensor of the invention, the filter for rejecting the excitation light at the wavelength λe need not be deposited on the front face of the assembly 10 of photodetectors 12, as shown in FIGS. 1 to 4, but could be deposited on the rear face, as shown in FIG. 5, i.e. on its face opposite from the face having the photodetectors 12. Under such circumstances, the silicon substrate is thinned down to about ten μm in order to avoid photons being absorbed by the silicon. Even after being thinned, the substrate forms extra thickness that moves the points of light away from the plane of the photodetectors which can lead to increasing the crosstalk interference signal. Computer processing of the data, based on deconvolution of the image including interference, can make it possible to eliminate the interference signal.

In the embodiment of FIG. 5, the absorbing layer 14 or the set of absorbing layers is covered by a layer 42 of p-doped silicon that is itself covered in a layer 44 of n-doped silicon, above which the photodetectors 12 are located. The biological probe deposition spots 16 are formed on the bottom face of the Bragg mirror or of the interference filter and they are illuminated by the excitation light at the wavelength λe. This makes it possible in particular to improve sensitivity by a factor of 2, since the assemblies 10 of CCD photodetectors illuminated on their front faces (beside the photodetectors 12) present reduced sensitivity in the visible spectrum because of photons being absorbed by the polysilicon transfer grids that are located at the photodetectors 12.

In all embodiments of the invention, the chromophores may be organic or inorganic nanocrystals incorporated in the surface layer of the biosensor, as described in document WO 2004/005590.

According to another characteristic of the invention, shown in FIG. 6, it is possible to form one or more openings 46, e.g. rectangular openings, in the top layer(s) 14, 20 of the biosensor for calibrating one or more of the following elements: Bragg mirror (or interference filter); absorbent layer; pre-deposited biological material; . . . . The surface of the biosensor is illuminated with the excitation light, and the signals delivered by the photodetectors 12 situated in register with the openings are compared with the signals delivered by the photodetectors situated away from the openings, for the purpose of calibrating the extent to which the excitation light is rejected by the Bragg mirror, by the absorbent layer, by the Bragg mirror and the absorbent layer together, etc. . . .

The openings 46 may be formed either in a single region, or else in different regions, occupying only a few percent of the useful surface of the biosensor.

As shown in FIG. 7, it is possible in the biosensor of the invention to make use of a matrix 10 of photodetectors 12a, 12b that are of different sizes. This makes it possible to deposit duplicate chromophores over pixels of different sizes in order to benefit from different dynamic ranges in the signals delivered by the pixels, which is advantageous for signals that are very weak or very strong.

In the variant embodiment shown in FIG. 8, a metallic film 48 is deposited on the surface of the biosensor over the above-mentioned rejection filter, the film 48 including openings 50 of very small size, of a dimension smaller than the wavelength of the light emitted by the chromophores. These openings define very small observation volumes (e.g. having a diameter of 150 nm) for detecting and observing individual chromophores in solutions at high concentration. These openings also amplify the light emitted by the chromophores that are to be found in their immediate vicinity.

In a variant, the metallic (or opaque) film 48 is deposited on the biosensor of FIG. 6, and the holes formed in the film are of dimensions that are greater and are in register with the openings 46 formed in the various layers of the rejection filter.

The biosensor of the invention can be used in conventional manner in stationary fluorescent solutions. However it can also be used in moving fluorescent solutions, in particular microfluidic circuits.

1. A method of fabricating an integrated detection biosensor, the biosensor comprising a substrate for carrying chromophore elements that emit light in response to light excitation at a given wavelength, and an assembly of photodetectors associated with the substrate to pick up the light the chromophore elements emit towards the inside of the substrate, the method comprising depositing thin layers on the assembly of photodetectors, the thin layers constituting the above-mentioned substrate together with a filter both for omnidirectional rejection of the chromophore element excitation light and for transmission of the light emitted by said elements, the filter presenting excitation light rejection of 10⁻⁶ or less and preferably about 10⁻⁸, and an autofluoresence level of 10⁻⁶ or less, the wavelength of the excitation light lying in the visible spectrum or in the near infrared.

2. A method according to claim 1, wherein the rejection filter includes at least one thin layer that is absorbent at the excitation wavelength.

3. A method according to claim 2, wherein the rejection filter also comprises a Bragg mirror made up of thin layers that are transparent at the wavelength emitted by the chromophore elements, having respective high and low refractive indices and placed in alternation.

4. A method according to claim 3, wherein the thin layers of the Bragg mirror present optical thickness that is substantially equal to one-fourth of the excitation wavelength.

5. A method according to claim 1, wherein the rejection filter comprises an interference filter made up of a series of superposed thin polymer layers having respective high and low refractive indices placed in alternation.

6. A method according to claim 1, wherein the rejection filter comprises a series of thin layers forming a Bragg mirror or an interference filter and covering at least one absorbent layer deposited on the assembly of photodetectors.

7. A method according to claim 1, wherein the rejection filter comprises a plurality of superposed absorbent thin layers of different kinds, in which a lower layer, closer to the photodetectors, is for absorbing the autofluorescence of a higher layer.

8. A method according to claim 2, wherein the absorbent thin layer(s) of the rejection filter have optical density of not less than about 6.4 at the excitation wavelength.

9. A method according to claim 1, wherein the thin layers of the rejection filter are made by a sol-gel method.

10. A method according to claim 1, wherein at least one thin layer that is absorbent at the excitation wavelength is deposited or formed on the assembly of photodetectors, and then the thin layers forming a Bragg mirror or an interference filter are deposited or formed in succession on the absorbent thin layer.

11. A method according to claim 1, wherein the rejection filter is formed on an initial substrate and is then transferred onto the assembly of photodetectors, the filter being located between the assembly of photodetectors and the initial substrate, the initial substrate subsequently being removed.

12. A method according to claim 11, wherein the rejection filter is fastened on the assembly of photodetectors by adhesion.

13. A method according to claim 11, wherein the rejection filter and the initial substrate form a flexible film.

14. A method according to claim 1, wherein the rejection filter comprises an absorbent film and a Bragg mirror or an interference filter that are placed together on the assembly of photodetectors.

15. A method according to claim 1, wherein the rejection filter comprises an absorbent film and a Bragg mirror or an interference filter that are placed separately on the assembly of photodetectors.

16. A method according to claim 1, wherein the absorbent layer is made by dissolving a dye in a solvent, mixing the dye solution with a solution of polyimide or butylcyclobenzene, depositing said mixture on a substrate or on the assembly of photodetectors, and annealing by passing the substrate or the assembly of photodetectors carrying the absorbent layer in a stove, said absorbent layer having thickness of about 10 μm or greater and optical density of not less than about 6 at the excitation wavelength.

17. A method according to claim 1, wherein probes optionally including fluorescent markers are subsequently deposited in spots on the rejection filter.

18. A method according to claim 17, wherein a buffer liquid containing a wetting agent is used for depositing probes on the rejection filter.

19. A method according to claim 17, wherein the biosensor carrying the probes is encapsulated in a cartridge or a package usable for hybridizing probes and having at least one inlet and one outlet for liquid interconnected by a channel extending over the surface of the filter carrying the probes, and at least one window for observing and/or illuminating the probes by the excitation light, the assembly of photodetectors having an electronic interface for connection to data processor means, and accessible via the rear face of the package or the cartridge.

20. A method according to claim 1, wherein the assembly of photodetectors is a matrix of photodetectors of CCD or CMOS type, having a front face carrying the filter for rejecting the excitation wavelength.

21. A method according to claim 1, wherein the assembly of photodetectors is a matrix of photodetectors of CCD or CMOS type, having a rear face carrying the filter for rejecting the excitation wavelength.

22. A method according to claim 1, wherein openings are formed in the layers of the rejection filter in register with the photodetectors for calibrating the rejection by said layers of the excitation light.

23. A method according to claim 1, wherein the matrix of photodetectors comprises photodetectors of different sizes.

24. A method according to claim 1, comprising placing a metallic film on the surface of the biosensor, the film including openings of a size smaller than the wavelength emitted by the chromophores.

25. A method of using a biosensor fabricated in accordance with the method of claim 1, comprising placing the biosensor in a stationary or moving fluorescent solution, and wherein the excitation wavelength for the chromophore elements lies in the visible spectrum or in the near infrared.
